(19) United States
(12) Patent Application Publication
(10) Pub. No.: US 2006/0263351 A1
(43) Pub. Date: Nov. 23, 2006

(54) Methods and compositions for protection against thrombolysis associated reperfusion injury

(75) Inventors: Eng H. Lo, Newton, MA (US); Xiaoying Wang, West Roxbury, MA (US); G. William Rebeck, Washington, DC (US); Ken Arai, Chiba-shi (JP)

Correspondence Address: WOLF GREENFIELD & SACKS, PC, FEDERAL RESERVE PLAZA, 600 ATLANTIC AVENUE, BOSTON, MA, 02210-2206, US

(73) Assignee: The General Hospital Corporation, Boston, MA (US)

(21) Appl. No.: 10/540,344

(22) PCT Filed: Dec. 22, 2003
(86) PCT No.: PCT/US03/40953

§ 371 Date: May 15, 2006

Related U.S. Application Data
(60) Provisional application No. 60/437,565, filed on Dec. 31, 2002.

(51) Int. Cl. A61K 39/395; A61K 38/54

(52) U.S. Cl. 424/133.1; 514/12; 424/145.1

(57) ABSTRACT

The invention relates to products and methods for reducing cerebral hemorrhage and edema, which can be negative side-effects of thrombolytic therapies with tissue plasminogen activator (tPA) and/or urokinase plasminogen activator (uPA).

### GOVERNMENT SUPPORT

This invention was made in part with government support under grant number NINDS R01-NS37074 from the National Institutes of Health (NIH). The government may have certain rights in this invention.

### FIELD OF THE INVENTION

The invention relates to methods and products for reducing cerebral hemorrhage and/or edema associated with thrombolytic therapy. The invention is useful for treating and preventing side effects of thrombolytic therapy with tissue plasminogen activator (tPA) and or urokinase plasminogen activator (uPA).

### BACKGROUND OF THE INVENTION

Cerebrovascular diseases occur predominately in the middle and late years of life. They cause approximately 200,000 deaths in the United States each year, as well as considerable neurologic disability. Categories of cerebrovascular diseases include ischemia-infarction and intracranial hemorrhage. Symptoms of most cerebrovascular diseases include an abrupt onset of a focal neurologic deficit, which may remain constant, or may improve or worsen. It is the abrupt onset of a nonconvulsive and focal neurologic deficit that defines a stroke, or cerebrovascular accident (CVA). (See 15th Edition Harrison's Principles of Internal Medicine, CD-ROM Version 1.0, 2001, McGraw-Hill).

Thrombolytic reperfusion with tissue plasminogen activator (tPA) is the only FDA-approved treatment for acute ischemic stroke. However, the benefits of reperfusion therapy can be negated by serious complications involving cerebral hemorrhage and edema (ECASS Study Group, JAMA 274:1017-1025, 1995; Hacke W, et. al., Neurology 53 (Suppl 4):s3-s15, 1999; Larrue V, et. al., Stroke 28:957-960, 1999; NINDS rt-PA Stroke Study Group, New Engl J Med 333:1581-1587, 1995; and NINDS rt-PA Stroke Study Group, Stroke 28:2109-2118, 1997). The mechanism of these deleterious side effects remains unclear, although it has been shown that stroke patients with higher MMP-9 levels in plasma have worse outcomes and more hemorrhagic transformation (Montaner, et. al., Stroke, 32:1759-1766, 2001; Montaner, et. al., Stroke, 32:2762-2767, 2001). Even with these results, a major gap remains in the understanding of the molecular and cellular mechanisms of tPA-induced hemorrhage and edema.

### SUMMARY OF THE INVENTION

We have discovered that deleterious side effects of tissue plasminogen activator (tPA) and urokinase plasminogen activator (uPA) thrombolytic therapy may be reduced by the administration of compounds that inhibit binding of tPA with low-density lipoprotein-receptor-related protein (LRP) receptor and compounds that inhibit binding of uPA with uPAR. These compounds are useful in methods to prevent and treat these side effects, which include, but are not limited to: cerebral hemorrhage and/or edema. We have discovered that methods and compositions that reduce the downstream signaling pathways triggered by the tPA-LRP/uPA-uPAR receptors (e.g. by blocking binding or interfering with downstream effects of binding) may prevent the deleterious upregulation of metalloproteinases (MMPs) and other related proteases.

According to one aspect of the invention, methods for reducing a side effect associated with thrombolytic therapy are provided. The methods include inhibiting binding of tissue plasminogen activator (tPA) administered to a subject to a low-density lipoprotein-receptor-related protein (LRP) receptor. In some embodiments, inhibiting binding of tPA to LRP includes administering to a subject in need of such treatment an amount of an agent that reduces tissue plasminogen activator (tPA) binding to a low-density lipoprotein-receptor-related protein (LRP) receptor effective to reduce the side effect, wherein the agent is administered before, simultaneously with, or after tPA treatment. In certain embodiments, the side effect associated with thrombolytic therapy is cerebral hemorrhage and/or edema. In some embodiments, the subject is human. In some embodiments, the thrombolytic therapy is the administration of tPA. In certain embodiments, the agent that reduces tPA binding to a LRP receptor is administered before tPA treatment. In other embodiments, the agent that reduces tPA binding to a LRP receptor is administered simultaneously with tpA treatment and in other embodiments, the agent that reduces tPA binding to a LRP receptor is administered after tPA treatment. In certain embodiments, the administration is intravenous administration. In some embodiments, the agent is an antibody or antigen-binding fragment thereof. In some embodiments, the subject is suspected or known to be at risk for a condition selected from the group consisting of ischemia, hemorrhage, edema, and brain injury. In other embodiments, the subject is suspected or known to have a condition selected from the group consisting of: ischemia, hemorrhage, edema, and brain injury. In certain embodiments, the subject is suspected or known to have had a condition selected from the group consisting of: ischemia, hemorrhage, edema, and brain injury.

According to yet another aspect of the invention, methods for reducing a side effect associated with thrombolytic therapy are provided. The methods include inhibiting binding of urokinase plasminogen activator (uPA) administered to a subject to a urokinase plasminogen activator receptor (uPAR). In some embodiments, inhibiting binding of uPA to uPAR includes administering to a subject in need of such treatment an amount of an agent that reduces urokinase plasminogen activator (uPA) binding to a urokinase plasminogen activator receptor (uPAR) effective to reduce the side effect, wherein the agent is administered before, simultaneously with, or after uPA treatment. In certain embodiments, the side effect associated with thrombolytic therapy is cerebral hemorrhage and/or edema. In some embodiments, the subject is human. In some embodiments, the thrombolytic therapy is the administration of uPA. In some embodiments, the agent that reduces uPA binding to a uPAR is administered before uPA treatment. In other embodiments, the agent that reduces uPA binding to uPAR is administered simultaneously with uPA treatment and in yet other embodiments, the agent that reduces uPA binding to a uPAR is administered after uPA treatment. In certain embodiments, the administration is intravenous administration. In some embodiments, the agent is an antibody or antigen-binding fragment thereof. In certain embodiments, the subject is suspected or known to be at risk for a condition selected from the group consisting of: ischemia, hemorrhage, edema, and brain injury. In other embodiments, the subject is suspected or known to have a condition selected from the group consisting of: ischemia, hemorrhage, edema, and brain injury. In some embodiments, the subject is suspected or known to have had a condition selected from the group consisting of: ischemia, hemorrhage, edema and brain injury.

According to another aspect of the invention, methods for reducing a side effect associated with thrombolytic therapy are provided. The methods include administering to a subject in need of such treatment an effective amount of an agent that interferes with downstream signaling cascades that lead from tissue plasminogen activator-low-density lipoprotein-receptor-related protein receptor (tPA-LRP) and/or urokinase plasminogen activator-urokinase plasminogen activator receptor (uPA-uPAR) to upregulation of matrix metalloproteinases (MMPs) and other related proteases that degrade neurovascular unit integrity. In some embodiments, the side effect is cerebral hemorrhage and/or edema.

According to another aspect of the invention, methods of identifying a candidate agent that modulates tissue plasminogen activator (tPA) binding to a low-density lipoprotein-receptor-related protein (LRP) receptor are provided. The methods include contacting an LRP receptor with tPA in the presence of a candidate agent, determining the level of binding of the LRP receptor with the tPA, and comparing the level of binding of LRP with tPA with a control level of binding of LRP and tPA not contacted with the candidate agent as a measure of the ability of the candidate agent to modulate tPA binding to LRP receptor. In some embodiments, modulate is to reduce. In other embodiments, modulate is to increase. In certain embodiments, the tPA is labeled with a detectable label. In some embodiments, the LRP receptor is labeled with a detectable label.

According to yet another aspect of the invention, methods of identifying a candidate agent that modulates urokinase plasminogen activator (uPA) binding to a urokinase plasminogen activator receptor (uPAR) are provided. The methods include contacting a uPAR with uPA in the presence of a candidate agent, determining the level of binding of the uPAR with the uPA, and comparing the level of binding of uPAR with uPA with a control level of binding of uPAR and uPA not contacted with the candidate agent as a measure of the ability of the candidate agent to modulate uPA binding to uPAR receptor. In some embodiments, modulate is to reduce. In other embodiments, modulate is to increase. In some embodiments, the uPA is labeled with a detectable label. In certain embodiments, the uPAR is labeled with a detectable label.

According to another aspect of the invention, methods of thrombolytic therapy are provided. The methods include administering to a subject in need of such treatment a combination of an effective amount of a thrombolytic agent and an effective amount of an inhibitor of the binding of the thrombolytic agent to its receptor, wherein the binding of the thrombolytic agent to its receptor results in an increase in matrix metalloproteinase expression. In some embodiments, the thrombolytic agent is tPA and its receptor is LRP receptor. In certain embodiments, the thrombolytic agent is uPA and its receptor is uPAR.

According to yet another aspect of the invention, methods of identifying a thrombolytic tissue plasminogen activator (tPA) variant with reduced binding to a low-density lipoprotein-receptor-related protein (LRP) receptor are provided. The methods include modifying a tPA molecule to prepare modified tPA molecules, testing the thrombolytic activity of the modified tPA molecules, selecting modified tPA molecules that retain thrombolytic activity (modified thrombolytic tPA molecules), contacting an LRP receptor with the modified thrombolytic tPA molecules, determining the level of binding of the LRP receptor with modified thrombolytic tPA molecules, and comparing the level of binding of LRP receptor by modified thrombolytic tPA molecules with a control level of binding of LRP receptor by unmodified tPA as an indication of reduced binding of the modified thrombolytic tPA molecules to LRP receptor. In some embodiments, the modification of the tPA molecule comprises one or more modifications selected from the group consisting of amino acid substitutions, amino acid deletions, and post-translational modifications.

According to yet another aspect of the invention, methods of identifying a thrombolytic urokinase plasminogen activator (uPA) variant with reduced binding to an urokinase plasminogen activator receptor (uPAR), are provided. The methods include modifying a uPA molecule to prepare modified uPA molecules, testing the thrombolytic activity of the modified uPA molecules, selecting modified uPA molecules that retain thrombolytic activity (modified thrombolytic uPA molecules), contacting an uPAR with the modified thrombolytic uPA molecules, determining the level of binding of the uPAR with modified thrombolytic uPA molecules, and comparing the level of binding of uPAR by modified thrombolytic uPA molecules with a control level of binding of uPAR by unmodified uPA as an indication of reduced binding of the modified thrombolytic uPA molecules to uPAR. In some embodiments, the modification of the uPA molecule comprises one or more modifications selected from the group consisting of amino acid substitutions, amino acid deletions, and post-translational modifications.

The use of the foregoing compounds and agents in the preparation of medicaments is also provided, particularly for use in treatment of thrombolysis-associated reperfusion injury. The use of the foregoing compounds and agents in the preparation of medicaments is also provided, particularly for use in treatment of ischemia, hemorrhage, edema, or brain injury.

### BRIEF DESCRIPTION OF THE DRAWINGS

FIG. 1 is a bar graph that shows result of treatment with the metalloproteinase (MMP) inhibitor. The results indicate that BB-94 significantly decreased tPA-induced hemorrhage in rat embolic focal ischemia. *P<0.05.

FIG. 2 shows a digitized image of zymograms and a corresponding histogram indicating effects on rat brain following embolic stroke. FIG. 2A shows representative rats treated with saline (sal) and tPA. N is normal brain. PC is a positive control comprising rat MMP-9 and MMP-2 standards. Note that in this zymogram, both pro-form and cleaved/active form of MMP-9 are detected. FIG. 2B is a histogram of densitometry results of the zymograms showing doubling of MMP-9 after tPA (N=5 per group, *P<0.05). FIG. 2C shows zymograms from rat brain after 2 hr focal ischemia induced mechanically. At 24 hrs, MMP-9 is amplified by tPA compared to saline-treated rats. PC=positive controls (rat MMP-9 and MMP-2).

FIG. 3 is a digitized image of gelatin zymography (FIG. 3A) and corresponding histogram (FIG. 3B) that demonstrate that upregulation in MMP-9 in brain at 24 hrs after permanent focal ischemia was reduced in tPA knockouts compared to wild-type C57B16 mice.

FIG. 4 shows histograms indicating infarction and edema results following focal cerebral ischemia. FIG. 4A shows ischemic lesion volumes at 24 hrs after 2 hr transient focal ischemia. *P<0.05. FIG. 4B shows fluorescent quantitation of Evans blue leakage in perfused brain at 20 hrs after 2 hrs transient focal ischemia. *P<0.05. Both infarction and edema are significantly reduced in MMP-9 knockout mice compared to wild-type littermates.

FIG. 5 shows digitized images of Western blots of LRP in cells subjected to hypoxia and re-oxygenation. Neurons and astrocytes were obtained from rat cortex. Endothelial cultures were obtained from a bovine cerebral microvessel endothelial cell line (Cell Systems Corp). All cells were exposed to hypoxia for 4 hrs, then re-oxygenated. N=normal cells. H=hypoxia. R4, R12 and R20 indicate re-oxygenated cells at 4, 12, and 20 hrs respectively. Protein concentrations were checked with the Bradford assay and equal loading was confirmed by stripping and reprobing for beta-actin. LRP levels in all three cell types are upregulated after acute hypoxia-re-oxygenation.

FIG. 6 shows digitized images of a Western blot and a photomicrographic image indicating the upregulation of LRP in rat brain after focal ischemia in vivo. FIG. 6A is a Western blot of LRP in rat brain at 24 hrs after filament or embolic focal ischemia. Homogenates from N=3 brains were combined per lane. FIG. 6B shows immunohistochemistry results showing upregulation of LRP co-localizing with NeuN-positive neurons and EBA-positive endothelial cells.

FIG. 7 shows digitized images of zymograms of neuron-astrocyte co-cultures from rat cerebral cortex and bovine cerebral microvessel endothelial cells. FIG. 7A shows results after 24 hr exposure to tPA. tPA increased MMP-9 secretion. PC indicates positive controls loaded with MMP-9 and MMP-2 standards. FIG. 7B and FIG. 7C are histograms showing dose-response of tPA-induced MMP-9 and MMP-2 (respectively) from human brain endothelial cells.

FIG. 8 shows a histogram indicating that tPA induced MMP-9 production in wild-type murine embryonic fibroblasts (MEF-1), but in LRP knockout cells (PEA-13), there was no significant response.

FIG. 9 shows digitized images of Western blots of phospho-ERK, total ERK (FIG. 9A), phospho-p38, and total p38 (FIG. 9B) from primary rat neuron cultures after exposure to 40 μg/ml tPA. Both ERK and p38 MAP kinase pathways are rapidly activated.

FIG. 10 provides digitized images of Western blots and zymograms. FIG. 10A indicates that ischemia-induced activation/phosphorylation of ERK is reduced in a dose-dependent manner by the inhibitor U0126. FIG. 10B shows zymograms indicating ischemic upregulation in MMP-9. Compared to saline treated controls, U0126 attenuated MMP-9 levels. N=normal brain. sal=ischemic rats treated with saline. U=ischemic rats treated with U0126. PC indicates positive controls loaded with rat MMP-9 and MMP-2 standards.

### DETAILED DESCRIPTION OF THE INVENTION

We have discovered that proteolysis of neurovascular substrates by the matrix metalloproteinase (MMP) family of zinc endopeptidases may be involved in the negative side effects of tPA and uPA therapy in patients. Previously, the effects of tPA in stroke were thought to mainly involve clot lysis, but, surprisingly, we have now identified novel tPA actions involving the low-density-lipoprotein receptor related protein (LRP) receptor, which responds rapidly to ischemia. Our results show that LRP receptor is upregulated after hypoxia in cultured neurons, astrocytes, and cerebral endothelial cells in vitro, and that after focal ischemia in vivo, LRP receptor is upregulated in rat brain.

tPA avidly binds LRP receptor, which associates with known signal transduction proteins. The LRP receptor belongs to a family that previously has been thought to comprise scavenging receptors involved in clearance not signal transduction. Surprisingly, our results indicate that the activation of the LRP receptor, for example, by tPA, can lead to upregulation of members of the matrix metalloproteinase (MMP) family of zinc endopeptidases by activating intracellular signaling pathways, for example through specific MAP kinase signaling pathways. In addition, our results suggest that methods of blocking the binding of tPA to LRP receptor or uPA to uPAR, a receptor that specifically binds uPA, will reduce the upregulation of MMPs. Thus, the invention includes methods and compositions for reducing the negative side-effects that are associated with tPA and uPA thrombolytic therapy-associated damage, e.g. hemorrhage and edema.

Our results indicate that cerebral ischemia upregulates the LRP receptor, which avidly binds tPA. Although not wishing to be bound by a particular theory, our data suggests that administration of tPA, i.e., in thrombolytic therapy, leads to not only clot lysis, but binding of the tPA to LRP receptors. This binding may trigger signaling pathways (e.g. the MAP kinase signaling pathway) that activate gene transcription of one or more MMPs, for example, is MMP-9. The resulting increased level of MMP-9 activity and/or increased levels of activity of other MMPs stimulated by tPA and/or uPA binding, then degrades critical substrates in the neurovascular unit, comprising vascular, astrocytic, and neuronal compartments.

Thus, we have discovered that the administration of tPA, eg. in thrombolytic therapy, triggers downstream signaling cascades that lead from the activation of LRP receptor by the tPA binding to the upregulation of MMPs and other related proteases (e.g., heparanases and other neutral or serine proteases) that degrade neurovascular unit integrity. Our results also indicate that a similar signaling mechanism is involved with the other major thrombolytic agent urokinase plasminogen activator (uPA). In the case of uPA thrombolytic therapy, the uPA binds to the uPAR and upregulates MMPs that degrade neurovascular substrates. Ultimately, these proteolytic actions of the MMPs weaken blood vessel integrity, and lead to leakage (cerebral edema) and rupture (cerebral hemorrhage).

The methods of the invention involve the administration of agents that reduce binding of tPA to LRP receptor and/or reduce the binding of uPA to uPAR and therefore, are useful to reduce or prevent the deleterious effects of the hMPs and other proteases that are upregulated by the administration of tPA or uPA. The compositions of the invention include molecules that reduce binding of the thrombolytic agents tPA and/or uPA to their receptors, (LRP receptor and/or uPAR, respectively), and inhibit deleterious upregulations of MMPs and other related proteases, thereby reducing the side effect damage of tPA and/or uPA thrombolytic therapy. The methods of the invention also involve the administration of agents that reduce the upregulation of MMPs and other such proteases by tPA/LRP and/or uPA/uPAR binding. Compositions of the invention include molecules that reduce the upregulation of MMPs and/or related proteases that results from administration of thrombolytic therapy.

In addition to the LRP receptor, other members of the family of receptors called the low density lipoprotein (LDL) receptors (Herz J. Neuron 29:571-581, 2001; Herz J, & Strickland D K, J Clin Invest 108:779-784, 2001; and Willnow T E, et al., Nature Cell Biol 1:E157-E162, 1999) may also be involved in the cascade of signals that leads to the negative side effects of tPA thrombolytic therapy, including cerebral hemorrhage and edema. Core members of this gene family includes the LDL receptor, the very low density lipoprotein (VLDL) receptor, ApoER2, MEGF7, LRP1B, megalin, and the low-density-lipoprotein Related Protein (LRP) receptor. The compositions of the invention include molecules that reduce binding of the thrombolytic agents tPA and/or uPA to these alternative receptors, thereby reducing the side effect damage of tPA and/or uPA thrombolytic therapy.

As used herein, the term “thrombolytic therapy” means therapy to prevent or treat a cerebral condition such as ischemia, stroke, brain trauma, and edema, by breaking up blood clots and allowing reperfusion of brain tissues. This type of therapy is known to those of skill in the art to include the administration of tissue plasminogen activator (tPA) and/or urokinase plasminogen activator (uPA) to a subject, which may be done in subjects suspected of undergoing an acute stroke. Additional disorders that may indicate a need for treatment using the methods and/or compositions of the invention, include brain injury, edema, ischemia, and/or hemorrhage. As used herein, the term: “subject” means any mammal, including, but not limited to: humans, non-human primates, cats, dogs, sheep, pigs, horses, cows, rodents such as mice, rats, etc., that may be in need of treatment with tPA or uPA.

There are several types of subjects for which the methods and compositions of the invention are applicable. One group of subjects includes subjects who known to be or are suspected to be in the process of undergoing a cerebral event, such as a stroke or brain injury, that may result in cerebral hemorrhage or edema. These are subjects to whom the compositions of the invention may be administered in conjunction with (e.g. simultaneously with) the uPA and/or uPA administered for thrombolytic therapy.

A second group of subjects for whom the methods and compositions may be applicable includes subjects who have previously had treatment with tPA or uPA and the molecules of the invention that reduce tPA binding to LRP receptor and/or uPA binding to uPAR, are given following a prior administration of tPA and/or uPA. The subsequent administration of the molecules of the invention may reduce the detrimental side effects of the tPA and/or uPA treatments. The temporal difference between the times of administration of the tPA and/or uPA and the molecules of the invention may any amount from 1 second through minutes or hours later. The amount of time can be any amount that still may result in an effective reduction in the binding between tPA and or uPA with LRP receptors and/or uPAR respectively.

A third group of subjects to whom the methods of the invention are applicable are subjects who may benefit from prophylactic treatment with a molecule that reduces the binding of tPA to LRP receptor and/or uPA to uPAR. For example prophylactic administration may be indicated for a subject known or believed to be at risk for a cerebral condition. This would include subjects who are considered to be likely to have a cerebral condition, e.g. stroke or brain injury. The determination to include a subject in this group may be based on family history, personal medical history, or diagnostic testing. This group may include subjects who have had a stroke at a previous time, may be at risk for injury, or may be someone identified using a diagnostic method known to those of skill in the art such as a cerebral imaging method, e.g. MRI or CT scan. This group includes subjects for whom a composition of the invention may be administered independent of and/or prior to the administration thrombolytic therapy with tPA or uPA.

The composition of the invention may include molecules that preferentially target neuronal tPA/LRP and/or neuronal uPA/uPAR interactions and binding effects. These compositions can be specifically targeted to neuronal tissue using various delivery methods, including, but not limited to: administration to neuronal tissue, the addition of targeting molecules to direct the compositions of the invention to neuronal tissues, and other methods. Additional methods to specifically target molecules and compositions of the invention to brain tissue and/or neuronal tissues are known to those of ordinary skill in the art.

The invention involves, in part, the administration of a compound that reduces specific binding of tPA to a LRP receptor and/or the administration of a compound that reduces specific binding of uPA to a uPAR. As used herein, the terms “reduce specific binding” and “reduction” mean to decrease the level or amount of binding of tPA to LRP receptor and/or uPA to uPAR, to a level or amount that is statistically significantly less than a control level of binding. In some cases, the reduction in the level of binding means the level of binding is reduced to zero, in other cases the reduction in the level of binding means that the level of binding will be significantly less than a control level, but above zero binding. A control level of binding of the tPA and/or uPA to the LRP or uPAR respectively, is the level of binding that represents the normal level of binding when a tpA polypeptide is contacted with LRP receptor and/or when uPA is contacted with uPAR. It will be understood by one of ordinary skill in the art that the control level of binding may be a predetermined value, which can take a variety of forms. It can be a single value, such as a median or mean. It can be established based upon comparative groups, such as in groups having normal levels of tPA or uPA binding to LRP receptor or uPAR respectively and groups having abnormal levels of tPA or uPA binding to LRP receptor or uPAR respectively.

As used herein, the term “cerebral condition” includes, but is not limited to disorders or dysfunctional conditions such as stroke, ischemia, hemorrhage, edema, or brain injury. As used herein, the term “cerebral hemorrhage” means bleeding in the brain. This may be caused by blood vessel rupture in the brain. As used herein, the term “edema” means fluid release in the brain, which may be leakage from blood vessels or cells and may cause swelling in the brain.

The invention relates in part to the administration of a binding-reduction molecule of the invention in an amount effective to treat or prevent cerebral hemorrhage and/or edema.

As used herein, the term: “binding-reduction molecule” means a molecule that inhibits or reduces the normal (control) level of binding between tPA and LRP receptor and/or uPA and uPAR, respectively. As used herein the term “agent” means a binding-reduction molecule of the invention. The binding-reduction molecules of the invention may include small molecules, chemicals, polypeptides, (for example, competitive ligands and antibodies, or antigen-binding fragments thereof), and may also include nucleic acids. For example, compositions of the invention may include nucleic acids that encode a molecule that reduces binding and fragments thereof, nucleic acids that bind to other nucleic acids, (e.g. for antisense or RNAi methods), or may be polypeptides that reduce the binding of tPA or uPA to LRP receptor and uPAR, respectively. Such polypeptides include, but are not limited to antagonists to the receptors or antibodies or antigen-binding fragments thereof.

The binding-reduction molecules of the invention also include molecules that bind to tPA or uPA and modulate the level of binding of the tPA with LRP receptor and/or uPA with uPAR. For example, the invention includes molecules that bind to tPA or uPA and modulate the level of binding of the tPA with LRP receptor and/or uPA with uPAR. These molecules do not reduce the therapeutic thrombolytic effect of the tPA or uPA administered or, it they do reduce the level of a therapeutic thrombolytic effect, they do not eliminate the therapeutic thrombolytic effect. In some embodiments, the compositions of the invention may include molecules that bind to tPA or uPA and enhance binding of the tPA with LRP receptor and/or uPA with uPAR. As described above herein, the molecules may be nucleic acid molecules, polypeptides, small molecules, or chemicals. These tPA or uPA molecules can be identified using the methods described herein for screening and characterizing the binding-reduction molecules or agents of the invention. In addition, their effectiveness in the methods of the invention can be determined using the assays provided herein, e.g. assays in which the level of binding between tPA or uPA with LRP receptor or uPAR, respectively, is determined both in the absence and presence of a modulatory molecule.

The invention also relates in some aspects to the identification and testing of candidate agents and molecules that can reduce the binding of tPA and uPA to LRP receptor and uPAR respectively. The binding-reduction polypeptides and fragments of the invention can be screened for reducing binding using the same type of assays as described herein (e.g. in the Examples section). Using such assays, the binding-reduction polypeptides and/or nucleic acid molecules that have the best inhibitory activity can be identified. It is understood that any mechanism of action described herein for the binding-reduction polypeptides and/or nucleic acids is not intended to be limiting, and the scope of the invention is not bound by any such mechanistic descriptions provided herein.

The binding-reduction molecules or agents of the invention also include small molecules and/or chemicals that reduce tPA or uPA binding to their respective receptors. The binding reduction molecules may be identified using the assays provided herein, including those in the Examples section. For example, a candidate agent or compound may be tested for its ability to reduce tPA or uPA binding to an LRP receptor or uPAR (e.g., an agent which selectively inhibits the level or effect of binding of activity of tPA or uPA with its respective binding partner). To test the ability of an agent or compound to reduce this specific binding, tPA and LRP receptor or uPA and uPAR may be contacted with a candidate binding-reduction compound or agent and the level of binding of tPA or uPA with their respective receptor can be compared to the level of binding of tPA or uPA with their respective receptors in the absence of a candidate binding-reduction compound or agent.

The invention further provides efficient methods of identifying pharmacological agents or lead compounds for agents and molecules that reduce the binding of tPA and/or uPA with LRP receptor and uPAR, respectively. Generally, the screening methods involve assaying for compounds which modulate (up- or down-regulate) the level of binding between tPA and/or uPA with LRP receptor and uPAR, respectively. As will be understood by one of ordinary skill in the art, the screening methods may measure level of binding between the molecules directly. In addition, screening methods may be utilized that measure a secondary effect of the binding of the tPA with LRP receptor or the binding of uPA with uPAR, for example the level of production of an MMP, e.g. MMP-9 in a cell or tissue sample. These secondary effects may also include the thrombolytic effect of tPA, which may be assayed to determine whether a candidate molecule reduces or interferes with the level of therapeutic thrombolytic activity of the tPA or uPA.

A wide variety of assays for pharmacological agents can be used in accordance with this aspect of the invention, including, labeled in vitro protein-protein binding assays, electrophoretic mobility shift assays, immunoassays, cell-based assays such as two- or three-hybrid screens, expression assays, etc. The assay mixture comprises a candidate pharmacological agent. Typically, a plurality of assay mixtures are run in parallel with different agent concentrations to obtain a different response to the various concentrations. Typically, one of these concentrations serves as a negative control, i.e., at zero concentration of agent or at a concentration of agent below the limits of assay detection.

Candidate agents useful in accordance with the invention encompass numerous chemical classes, although typically they are organic compounds. Preferably, the candidate pharmacological agents are small organic compounds, i.e., those having a molecular weight of more than 50 yet less than about 2500, preferably less than about 1000 and, more preferably, less than about 500. Candidate agents comprise functional chemical groups necessary for structural interactions with proteins and/or nucleic acid molecules, and typically include at least an amine, carbonyl, hydroxyl or carboxyl group, preferably at least two of the functional chemical groups and more preferably at least three of the functional chemical groups. The candidate agents can comprise cyclic carbon or heterocyclic structure and/or aromatic or polyaromatic structures substituted with one or more of the above-identified functional groups. Candidate agents also can be biomolecules such as peptides, saccharides, fatty acids, sterols, isoprenoids, purines, pyrimidines, derivatives or structural analogs of the above, or combinations thereof and the like. Where the agent is a nucleic acid molecule, the agent typically is a DNA or RNA molecule, although modified nucleic acid molecules as defined herein are also contemplated.

It is contemplated that cell-based assays as described herein can be performed using cell samples and/or cultured cells. Biopsy cells and tissues as well as cell lines grown in culture are useful in the methods of the invention.

Candidate agents are obtained from a wide variety of sources including libraries of synthetic or natural compounds. For example, numerous means are available for random and directed synthesis of a wide variety of organic compounds and biomolecules, including expression of randomized oligonucleotides, synthetic organic combinatorial libraries, phage display libraries of random peptides, and the like. Alternatively, libraries of natural compounds in the form of bacterial, fungal, plant and animal extracts are available or readily produced. Additionally, natural and synthetically produced libraries and compounds can be readily be modified through conventional chemical, physical, and biochemical means. Further, known pharmacological agents may be subjected to directed or random chemical modifications such as acylation, alkylation, esterification, amidification, etc. to produce structural analogs of the agents.

A variety of other reagents also can be included in the mixture. These include reagents such as salts, buffers, neutral proteins (e.g., albumin), detergents, etc. which may be used to facilitate optimal protein-protein and/or protein-nucleic acid binding. Such a reagent may also reduce non-specific or background interactions of the reaction components. Other reagents that improve the efficiency of the assay such as protease inhibitors, nuclease inhibitors, antimicrobial agents, and the like may also be used.

An exemplary binding assay is described herein, which may be used to identify candidate agents that modulate the binding of tPA to LPR receptor or uPA to uPAR. In general, the mixture of the foregoing assay materials is incubated under conditions whereby, but for the presence of the candidate pharmacological agent, the tPA binds to LRP receptor and/or uPA binds to uPAR, although in some embodiments the candidate agent may be one that increases the binding between tPA and uPA and their respective receptors. The order of addition of components, incubation temperature, time of incubation, and other parameters of the assay may be readily determined. Such experimentation merely involves optimization of the assay parameters, not the fundamental composition of the assay. Incubation temperatures typically are between 4° C. and 40° C. Incubation times preferably are minimized to facilitate rapid, high throughput screening, and typically are between 0.1 and 10 hours.

After incubation, the presence or absence of specific binding between the tPA and LRP and/or uPA and uPAR is detected by any convenient method available to the user. For cell-free binding type assays, a separation step is often used to separate bound from unbound components. The separation step may be accomplished in a variety of ways. Conveniently, at least one of the components is immobilized on a solid substrate, from which the unbound components may be easily separated. The solid substrate can be made of a wide variety of materials and in a wide variety of shapes, e.g., microtiter plate, microbead, dipstick, resin particle, etc. The substrate preferably is chosen to maximum signal to noise ratios, primarily to minimize background binding, as well as for ease of separation and cost.

Separation may be effected for example, by removing a bead or dipstick from a reservoir, emptying or diluting a reservoir such as a microtiter plate well, rinsing a bead, particle, chromatographic column or filter with a wash solution or solvent. The separation step preferably includes multiple rinses or washes. For example, when the solid substrate is a microtiter plate, the wells may be washed several times with a washing solution, which typically includes those components of the incubation mixture that do not participate in specific bindings such as salts, buffer, detergent, non-specific protein, etc. Where the solid substrate is a magnetic bead, the beads may be washed one or more times with a washing solution and isolated using a magnet.

Detection may be effected in any convenient way for cell-based assays such as two- or three-hybrid screens. For cell-free binding assays, one of the components usually comprises, or is coupled to, a detectable label. A wide variety of labels can be used, such as those that provide direct detection (e.g., radioactivity, luminescence, optical or electron density, etc.) or indirect detection (e.g., epitope tag such as the FLAG epitope, enzyme tag such as horse-radish peroxidase, etc.). The label may be bound to a tPA or uPA or to a LRP receptor or a uPAR or may be incorporated in to the candidate agent.

A variety of methods may be used to detect the label, depending on the nature of the label and other assay components. For example, the label may be detected while bound to the solid substrate or subsequent to separation from the solid substrate. Labels may be directly detected through optical or electron density, radioactive emissions, nonradiative energy transfers, etc. or indirectly detected with antibody conjugates, strepavidin-biotin conjugates, etc. Methods for detecting the labels are well known in the art.

In some aspects of the invention, the agents and compounds are isolated nucleic acid molecules, that are useful for practicing the invention. In other embodiments, the compositions include isolated polypeptides, that are encoded by the above-described nucleic acid molecules. The polypeptides used in the methods of the invention embrace polypeptides as well as polypeptide fragments. The binding-reduction polypeptides of the invention include fragments, (i.e. pieces) of binding-reduction molecules. These fragments are shorter than the full-length binding-reduction molecules.

As used herein with respect to nucleic acids, the term “isolated” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art. An isolated nucleic acid molecule as used herein is not a naturally occurring chromosome.

The polypeptides useful for practicing the invention can be isolated from biological samples including tissue or cell homogenates, and can also be expressed recombinantly in a variety of prokaryotic and eukaryotic expression systems by constructing an expression vector appropriate to the expression system, introducing the expression vector into the expression system, and isolating the recombinantly expressed protein. Short polypeptides, also can be synthesized chemically using well-established methods of peptide synthesis.

Thus, as used herein with respect to polypeptides, “isolated” means separated from its native environment and present in sufficient quantity to permit its identification or use. Isolated, when referring to a polypeptide, means, for example: (i) selectively produced by expression of a recombinant nucleic acid or (ii) purified as by chromatography or electrophoresis.

Isolated polypeptides may, but need not be, substantially pure. The term “substantially pure” means that the polypeptides are essentially free of other substances with which they may be found in nature or in in vivo systems to an extent practical and appropriate for their intended use. Substantially pure polypeptides may be produced by techniques well known in the art. Because an isolated polypeptide may be admixed with a pharmaceutically acceptable carrier in a pharmaceutical preparation, the polypeptide may comprise only a small percentage by weight of the preparation. The polypeptide is nonetheless isolated in that it has been separated from the substances with which it may be associated in living systems, i.e. isolated from other polypeptides.

Homologs and alleles of the binding-reduction polypeptides of the invention can be identified by conventional techniques. As used herein, a homolog to a binding-reduction polypeptide is a polypeptide from a human or other animal that has a high degree of structural similarity to the identified binding-reduction polypeptide, e.g., at least 95% amino acid sequence identity.

Identification of human and other organism homologs of binding-reduction polypeptides will be familiar to those of skill in the art. In general, nucleic acid hybridization is a suitable method for identification of homologous sequences of another species (e.g., human, cow, sheep), which correspond to a known sequence. Standard nucleic acid hybridization procedures can be used to identify related nucleic acid sequences of selected percent identity. For example, one can construct a library of cDNAs reverse transcribed from the mRNA of a selected tissue and use the nucleic acids that encode binding-reduction polypeptides identified herein to screen the library for related nucleotide sequences. The screening preferably is performed using high-stringency conditions to identify those sequences that are closely related by sequence identity. Nucleic acids so identified can be translated into polypeptides and the polypeptides can be tested for a binding-reduction functional activity, (e.g. a reduction of tPA and/or uPA-induced MMP activity).

The term “high stringency” as used herein refers to parameters with which the art is familiar. Nucleic acid hybridization parameters may be found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. More specifically, high-stringency conditions, as used herein, refers, for example, to hybridization at 65° C. in hybridization buffer (3.5×SSC, 0.02% Ficoll, 0.02% polyvinyl pyrrolidone, 0.02% Bovine Serum Albumin, 2.5 mM NaH₂PO₄ (pH7), 0.5% SDS, 2 mM EDTA). SSC is 0.15M sodium chloride/0.015M sodium citrate, pH7; SDS is sodium dodecyl sulphate; and EDTA is ethylenediaminetetracetic acid. After hybridization, the membrane upon which the DNA is transferred is washed, for example, in 2×SSC at room temperature and then at 0.1-0.5×SSC/0.1×SDS at temperatures up to 68° C.

There are other conditions, reagents, and so forth that can be used, which result in a similar degree of stringency. The skilled artisan will be familiar with such conditions, and thus they are not given here. It will be understood, however, that the skilled artisan will be able to manipulate the conditions in a manner to permit the clear identification of homologs and alleles of binding-reduction polypeptide nucleic acids of the invention (e.g., by using lower stringency conditions). The skilled artisan also is familiar with the methodology for screening cells and libraries for expression of such molecules, which then are routinely isolated, followed by isolation of the pertinent nucleic acid molecules and sequencing.

In general, binding-reduction homologs and alleles typically will share at least 90% nucleotide identity and/or at least 95% amino acid identity to the sequences of binding-reduction polypeptides or fragments thereof, and precursors thereof. Nucleic acid and polypeptides, respectively, in some instances will share at least 95% nucleotide identity and/or at least 97% amino acid identity, and in other instances will share at least 97% nucleotide identity and/or at least 99% amino acid identity. The percent identity can be calculated using various, publicly available software tools developed by NCBI (Bethesda, Md.) that can be obtained through the internet. Exemplary tools include the BLAST system available from the website of the National Center for Biotechnology Information (NCBI) at the National Institutes of Health. Pairwise and ClustalW alignments (BLOSUM30 matrix setting) as well as Kyte-Doolittle hydropathic analysis can be obtained using the MacVector sequence analysis software (Oxford Molecular Group). Watson-Crick complements of the foregoing nucleic acids also are embraced by the invention.

In screening for binding-reduction polypeptide genes, a Southern blot may be performed using the foregoing conditions, together with a detectably labeled probe (e.g. radioactive or chemiluminescent probes). After washing the membrane to which the DNA is finally transferred, the membrane can be placed against X-ray film or a phosphorimager to detect the radioactive or chemiluminescent signal. In screening for the expression of binding-reduction polypeptide nucleic acids, Northern blot hybridizations using the foregoing conditions can be performed. Amplification protocols such as polymerase chain reaction using primers that hybridize to the sequences presented also can be used for detection of the binding-reduction polypeptide genes or expression thereof.

Identification of related sequences can also be achieved using polymerase chain reaction (PCR) and other amplification techniques suitable for cloning related nucleic acid sequences. Preferably, PCR primers are selected to amplify portions of a nucleic acid sequence believed to be conserved (e.g., a binding domain, etc.). Again, nucleic acids are preferably amplified from a tissue-specific library.

The invention also includes degenerate nucleic acids that include alternative codons to those present in the native materials. For example, serine residues are encoded by the codons TCA, AGT, TCC, TCG, TCT and AGC. Each of the six codons is equivalent for the purposes of encoding a serine residue. Thus, it will be apparent to one of ordinary skill in the art that any of the serine-encoding nucleotide triplets may be employed to direct the protein synthesis apparatus, in vitro or in vivo, to incorporate a serine residue into an elongating binding-reduction polypeptide. Similarly, nucleotide sequence triplets which encode other amino acid residues include, but are not limited to: CCA, CCC, CCG, and CCT (proline codons); CGA, CGC, CGG, CGT, AGA, and AGG (arginine codons); ACA, ACC, ACG, and ACT (threonine codons); AAC and AAT (asparagine codons); and ATA, ATC, and ATT (isoleucine codons). Other amino acid residues may be encoded similarly by multiple nucleotide sequences. Thus, the invention embraces degenerate nucleic acids that differ from the biologically isolated nucleic acids in codon sequence due to the degeneracy of the genetic code.

The invention also provides modified nucleic acid molecules, which include additions, substitutions and deletions of one or more nucleotides (preferably 1-20 nucleotides that are useful for practicing the invention). In preferred embodiments, these modified nucleic acid molecules and/or the polypeptides they encode retain at least one activity or function of the unmodified nucleic acid molecule and/or the polypeptides, such as binding reduction and/or inhibition of tPA or uPA-associated increases in MMP, etc. In certain embodiments, the modified nucleic acid molecules encode modified polypeptides, preferably polypeptides having conservative amino acid substitutions as are described elsewhere herein. The modified nucleic acid molecules are structurally related to the unmodified nucleic acid molecules and in preferred embodiments are sufficiently structurally related to the unmodified nucleic acid molecules so that the modified and unmodified nucleic acid molecules hybridize under high stringency conditions known to one of skill in the art.

For example, modified nucleic acid molecules that encode polypeptides having single amino acid changes can be prepared. Each of these nucleic acid molecules can have one, two, or three nucleotide substitutions exclusive of nucleotide changes corresponding to the degeneracy of the genetic code as described herein. Likewise, modified nucleic acid molecules that encode polypeptides having two amino acid changes can be prepared which have, e.g., 2-6 nucleotide changes. Numerous modified nucleic acid molecules like these will be readily envisioned by one of skill in the art, including for example, substitutions of nucleotides in codons encoding amino acids 2 and 3, 2 and 4, 2 and 5, 2 and 6, and so on. In the foregoing example, each combination of two amino acids is included in the set of modified nucleic acid molecules, as well as all nucleotide substitutions which code for the amino acid substitutions. Additional nucleic acid molecules that encode polypeptides having additional substitutions (i.e., 3 or more), additions or deletions (e.g., by introduction of a stop codon or a splice site(s)) also can be prepared and are embraced by the invention as readily envisioned by one of ordinary skill in the art. Any of the foregoing nucleic acids or polypeptides can be tested by routine experimentation for retention of structural relation or activity to the nucleic acids and/or polypeptides disclosed herein.

Fragments can be used as probes in Southern and Northern blot assays to identify such nucleic acids, or can be used in amplification assays such as those employing PCR. As known to those skilled in the art, large probes such as 200, 250, 300 or more nucleotides are preferred for certain uses such as Southern and Northern blots, while smaller fragments will be preferred for uses such as PCR. Fragments also can be used to produce fusion proteins for generating antibodies or determining binding of the polypeptide fragments, or for generating immunoassay components. Likewise, fragments can be employed to produce nonfused fragments of the binding-reduction polypeptide, useful, for example, in the preparation of antibodies, and in immunoassays. The antibodies can be used, for example, to identify specific epitopes in LRP receptor polypeptides or uPAR polypeptides that are responsible for binding modulation.

Fragments of a polypeptide preferably are those fragments that retain a distinct functional capability of the polypeptide. Functional capabilities that can be retained in a fragment of a polypeptide include the ability to inhibit binding of tPA or uPA with LRP or uPAR respectively. As will be recognized by those skilled in the art, the size of the fragment will depend upon factors such as whether the fragment is of sufficient size to binding. Thus, some fragments of binding-reduction polypeptides will consist of longer segments while others will consist of shorter segments, (e.g. 5, 6, 7, 8, 9, 10, 11 or 12 or more amino acids long, including each integer up to the full length of the binding-reduction polypeptide). Those skilled in the art are well versed in methods for selecting functional fragments of polypeptides.

The skilled artisan will also realize that conservative amino acid substitutions may be made in binding-reduction polypeptides to provide functionally equivalent variants, or homologs of the foregoing polypeptides, i.e, the variants retain the functional capabilities of the binding-reduction polypeptides. As used herein, a “conservative amino acid substitution” refers to an amino acid substitution that does not alter the relative charge or size characteristics of the protein in which the amino acid substitution is made. Variants can be prepared according to methods for altering polypeptide sequence known to one of ordinary skill in the art such as are found in references that compile such methods, e.g. Molecular Cloning: A Laboratory Manual, J. Sambrook, et al., eds., Second Edition, Cold Spring Harbor Laboratory Press, Cold Spring Harbor, N.Y., 1989, or Current Protocols in Molecular Biology, F. M. Ausubel, et al., eds., John Wiley & Sons, Inc., New York. Exemplary functionally equivalent variants or homologs of the binding-reduction polypeptides include conservative amino acid substitutions in the amino acid sequences of proteins disclosed herein. Conservative substitutions of amino acids include substitutions made amongst amino acids within the following groups: (a) M, I, L, V; (b) F, Y, W; (c) K, R, H; (d) A, G; (e) S, T; (f) Q, N; and (g) E, D. For example, upon determining that a peptide is a binding-reduction-equivalent polypeptide, one can make conservative amino acid substitutions to the amino acid sequence of the peptide, and still have the polypeptide retain its specific binding-reduction and/or characteristics.

Conservative amino-acid substitutions in the amino acid sequence of binding-reduction polypeptides to produce functionally equivalent variants of binding-reduction polypeptides typically are made by alteration of a nucleic acid encoding binding-reduction polypeptides. Such substitutions can be made by a variety of methods known to one of ordinary skill in the art. For example, amino acid substitutions may be made by PCR-directed mutation, site-directed mutagenesis according to the method of Kunkel (Kunkel, Proc. Nat. Acad. Sci. U.S.A. 82: 488-492, 1985), or by chemical synthesis of a gene encoding a binding-reduction polypeptide. Where amino acid substitutions are made to a small unique fragment of a binding-reduction polypeptide, the substitutions can be made by directly synthesizing the peptide. The activity of functionally equivalent fragments of binding-reduction polypeptides can be tested by cloning the gene encoding the altered binding-reduction polypeptide into a bacterial or mammalian expression vector, introducing the vector into an appropriate host cell, expressing the altered polypeptide, and testing for a functional capability of the binding-reduction polypeptides as disclosed herein. Peptides that are chemically synthesized can be tested directly for function, e.g., for inhibiting binding to the LRP receptor or uPAR.

Polypeptides, nucleic acids, small molecules, and chemicals that modulate tPA binding to LRP receptor and uPA binding to uPAR, can be modified as described herein. These modified agents or compounds can be tested for their ability to modulate binding between tPA and LRP receptor and/or uPA and uPAR using the assay methods provided herein. The screening assays described can be used to test any modification of a molecule that has been found to modulate the binding of tPA and uPA to LRP receptor and uPAR, respectively, and thus, the efficacy of any modified agent or binding-reduction molecule can be determined using the methods provided.

In some embodiments of the invention, the binding-reduction polypeptide that selectively reduce binding of tPA or uPA to a LRP receptor or uPAR, respectively, is an antibody or antibody fragment, more preferably, an Fab or F(ab)₂ fragment of an antibody. Typically, the fragment includes a complementarity-determining region (CDR) that is selective for the LRP receptor or uPAR. Any of the various types of antibodies can be used for this purpose, including monoclonal antibodies, anti-peptide antibodies, humanized antibodies, single chain antibodies, and chimeric antibodies.

Thus, the invention provides agents that bind to LRP receptor or uPAR, and interferes with binding of tPA or uPA to their respective receptors. Additionally, the invention provides agents that bind to tPA or uPA and interfere with binding of each to its respective receptor without eliminating the therapeutic thrombolytic effect of the tPA or uPA. The activity of antibodies and antigen-binding fragments thereof to modulate binding of tPA with LRP receptor and/or uPA with uPAR, can be determined using the assays provided herein. Such binding partners can also be used in screening assays to detect the presence or absence of a LRP receptor or uPAR and in purification protocols to isolate such LRP receptors or uPARs. Likewise, such binding partners can be used to selectively target drugs, toxins or other molecules to cells which express LRP receptors or uPARs. In this manner, cells present in culture or in the brain tissues that express LRP receptors or uPARs can be treated with additional therapeutic compounds. Such agents also can be used to inhibit the native activity of the LRP receptors or uPARs, for example, by reducing the binding of tPA and/or uPA to such polypeptides for the prevention and/or treatment of cerebral hemorrhage or edema.

The invention, therefore, provides antibodies or fragments of antibodies having the ability to selectively bind to LRP receptors or uPARs, and preferably to unique fragments thereof. Antibodies include polyclonal, monoclonal, and chimeric antibodies, prepared, e.g., according to conventional methodology.

The antibodies of the present invention thus are prepared by any of a variety of methods, including administering protein, fragments of protein, cells expressing the protein or fragments thereof and the like to an animal to induce polyclonal antibodies. The production of monoclonal antibodies is according to techniques well known in the art. As detailed herein, such antibodies may be used for example to identify tissues expressing protein or to purify protein. Antibodies also may be coupled to specific labeling agents for imaging or to therapeutic agents, and so forth. Chemotherapeutic and radiotherapeutic agents useful in methods of the invention are known to those skilled in the art.

Significantly, as is well known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, I. (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)₂ fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Proceeding further, Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity-determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well established in the art that the non-CDR regions of a mammalian antibody may be replaced with similar regions of nonspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. (see, e.g., U.S. Pat Nos. 4,816,567, 5,225,539, 5,585,089, 5,693,762, and 5,859,205). Thus, for example, PCT International Publication Number WO 92/04381 teaches the production and use of humanized murine RSV antibodies in which at least a portion of the murine FR regions have been replaced by FR regions of human origin. Such antibodies, including fragments of intact antibodies with antigen-binding ability, are often referred to as “chimeric” antibodies.

Fully human monoclonal antibodies also can be prepared by immunizing mice transgenic for large portions of human immunoglobulin heavy and light chain loci. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans.

Thus, as will be apparent to one of ordinary skill in the art, the present invention also provides for F(ab′)₂, Fab, Fv and Fd fragments; chimeric antibodies in which the Fc and/or FR and/or CDR1 and/or CDR2 and/or light chain CDR3 regions have been replaced by homologous human or non-human sequences; chimeric F(ab′)₂ fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. Thus, the anti-peptide approach is another methodology that can be used to generate new antibodies.

The invention involves polypeptides of numerous size and type that bind specifically to LRP receptor proteins and/or uPAR proteins. These polypeptides may be derived also from sources other than antibody technology. For example, such polypeptide binding agents can be provided by degenerate peptide libraries which can be readily prepared in solution, in immobilized form or as phage display libraries. Combinatorial libraries also can be synthesized of peptides containing one or more amino acids. Libraries further can be synthesized of peptides and non-peptide synthetic moieties.

Phage display can be particularly effective in identifying binding peptides useful according to the invention. Briefly, one prepares a phage library (using e.g. m13, fd, or lambda phage), displaying inserts from 4 to about 80 amino acid residues using conventional procedures. The inserts may represent a completely degenerate or biased array. One then can select phage-bearing inserts which bind to a LRP receptor protein or a uPAR protein. This process can be repeated through several cycles of reselection of phage that bind to a LRP receptor protein or a uPAR protein. Repeated rounds lead to enrichment of phage bearing particular sequences. DNA sequence analysis can be conducted to identify the sequences of the expressed polypeptides. The minimal linear portion of the sequence that binds to the LRP receptor protein or the uPAR protein can be determined. One can repeat the procedure using a biased library containing inserts containing part or all of the minimal linear portion plus one or more additional degenerate residues upstream or downstream thereof. Thus, the LRP receptor or uPAR proteins of the invention can be used to screen peptide libraries, including phage display libraries, to identify and select peptide binding partners of the LRP receptor or uPAR proteins and assays provided herein can be utilized to determine whether the peptide binding partners are binding-reduction polypeptides useful in the invention. Such molecules can be used, as described, for screening assays, for diagnostic assays, for purification protocols or for targeting drugs, and/or labeling agents (e.g. radioisotopes, fluorescent molecules, etc.) to cells that express LRP receptors or uPAR proteins.

As detailed herein, the foregoing antibodies and other binding molecules may be coupled to specific diagnostic labeling agents for imaging of cells and tissues with LRP receptors or uPAR expression or to therapeutically useful agents according to standard coupling procedures. Diagnostic agents include, but are not limited to, barium sulfate, iocetamic acid, iopanoic acid, ipodate calcium, diatrizoate sodium, diatrizoate meglumine, metrizamide, tyropanoate sodium and radiodiagnostics including positron emitters such as fluorine-18 and carbon-11, gamma emitters such as iodine-123, technitium-99m, iodine-131 and indium-111, nuclides for nuclear magnetic resonance such as fluorine and gadolinium. Other diagnostic agents useful in the invention will be apparent to one of ordinary skill in the art.

The invention also relates in some aspects to the use of tPA and/or uPA variants that maintain a sufficient level of therapeutic thrombolytic activity for effective therapy, yet have a reduced ability to upregulate MMPs and other proteases after thrombolysis in stroke. The variants of the invention may exhibit a reduced affinity for binding with the LRP receptor and/or uPAR, respectively. The parameters of the tPA and uPA molecules that may be modified include sequence and structural features such as those described herein.

Thus, in accordance with the invention, tissue plasminogen activator (alteplase, tPA) and urokinase can be modified to create variant molecules that retain thrombolytic activity. The person of skill in the art is familiar with methods for modifying these molecules. For example, various modified forms of tPA (“modified tPA”) have been characterized and are known to those skilled in the art. Modified tPA includes, but is not limited to, variants having deleted or substituted amino acids or domains, variants conjugated to other molecules, and variants having modified glycosylation. For example, PCT Publication No. WO93/24635 discloses tPA variants having an extra glycosylation site at any of the amino acid positions 103-105 and the native glycosylation site removed at position 117 of the native human tPA. The amino acid number refers to the amino acid in that position of the mature, wild-type tPA polypeptide as disclosed in U.S. Pat. No. 4,766,075. The disclosed variants may also include at least one amino acid substituted in the 296-299 position with alanine and/or a substitution of the amino acids at positions 274-277 of wild type tPA (phenylalanine, arginine, isoleucine, lysine) with leucine, histidine, serine, and threonine, respectively. Triple mutants of tPA also are disclosed, including the specific molecule: T103N, N117Q, KHRR (296-299) AAAA t-PA (TNK t-PA). EP 352,119 discloses vampire bat tPAs (Bat-PAs (H), (I), and (L)). Vampire bat-PAs are variants of native tPA having a variety of sequence modifications. Suzuki et al., (J. Cardiovasc. Pharmacal. 22:834-840, 1993) disclose tPA variants in which a cysteine at position 84 of the growth factor domain of native tPA is replaced by serine (C84S tPA). Although this variant retains the functional activity of native tPA, it has been shown to have a longer in vivo half life than native tPA.

Variants of tPA have been developed which retain tPA functionality but have reduced clearance rates. These variants include tPA molecules with deleted amino acids or domains, such as those described by Johannessen et al. (Throm. Haemostas. 63:54-59, 1990) and Sobel et al. (Circulation 81:1362-73, 1990); tPA molecules which have amino acid substitutions in the regions of 63-72 and 42-49, such as those described by Ahem et al. (J. Biol. Chem. 265:5540, 1990); and tPA molecules which have a glutamic acid substituted for the arginine at position 275 of the native t-PA molecule such as that described by Hotchkiss et al. (Throm. Haemostas. 55:491, 1987). tPA molecules conjugated to other molecules have also been found to have decreased clearance rates. For example, conjugation of tPA to polyethylene glycol has been shown to reduce the clearance rate of tPA, as disclosed in EP-A304,311. Conjugation of a tPA molecule to a monoclonal antibody has been shown to increase the half-life of tPA in vivo (EP A339,505).

Modification of glycosylation on native tPA has also been found to have an effect on clearance rates of tPA. PCT application WO89/11531 discloses several tPA variants having additional glycosylation sites, which also have decreased clearance rates. Other research has described tPA variants with reduced glycosylation, which also exhibit decreased clearance rates (Martin et al., Fibrinolysis 4:9, 1990). Each of the above references is hereby incorporated by reference.

These and other modified tPA and urokinase molecules can be tested for activities consistent with the invention. Moreover, the procedures used to prepare these known tPA variants can be used to prepare additional tPA and urokinase variants. Such variants are tested for reduced binding to LRP and uPAR, respectively, as compared to non-modified molecules.

Additional compounds can be administered to a subject in addition to the tPA/uPA binding antagonists and/or tPA/uPA variants that have reduced binding to LRP/uPAR. For example antithrombotic compounds and/or neuroprotective agents can be administered prior to, substantially with, or after the tPA and/or uPA. Antithrombotics include anagrelide hydrochloride; bivalirudin; dalteparin sodium; danaparoid sodium; dazoxiben hydrochloride; efegatran sulfate; enoxaparin sodium; ifetroban; ifetroban sodium; tinzaparin sodium; and trifenagrel. Neuroprotective agents include dizocilpine maleate. Other useful agents will be known to one of ordinary skill in the art in the medical arts.

According to a further aspect of the invention, pharmaceutical compositions containing the nucleic acid molecules, proteins, and binding polypeptides of the invention are provided. The pharmaceutical compositions contain any of the therapeutic agents described herein, e.g. the binding-reduction molecules, in a pharmaceutically acceptable carrier. Thus, in a related aspect, the invention provides a method for forming a medicament that involves placing a therapeutically effective amount of the therapeutic agent in the pharmaceutically acceptable carrier to form one or more doses.

When used therapeutically, the binding-reduction molecules of the invention are administered in therapeutically effective amounts. In general, a therapeutically effective amount means that amount necessary to delay the onset of, inhibit the progression of, or halt altogether the particular condition being treated. Generally, a therapeutically effective amount will vary with the subject's age, condition, and sex, as well as the nature and extent of the condition in the subject, all of which can be determined by one of ordinary skill in the art.

The binding-reduction molecule dosage may be adjusted by the individual physician or veterinarian, particularly in the event of any complication. A therapeutically effective amount typically varies from 0.01 mg/kg to about 1000 mg/kg, preferably from about 0.1 mg/kg to about 200 mg/kg, and most preferably from about 0.2 mg/kg to about 20 mg/kg, in one or more dose administrations daily, for one or more days.

The therapeutically effective amount of the binding-reduction molecule is that amount effective to binding of tPA or uPA to their respective receptors, eg. LRP receptor and/or uPAR, and reduces the cerebral hemorrhage and or edema associated with tPA or uPA therapy. The presence and/or level of cerebral hemorrhage and or edema can be determined using methods known to those of skill in the art. For example, imaging methods can be used to determine the level of cerebral bleeding or the absence of bleeding, etc. These types of tests, as well as others known to those of ordinary skill in the medical arts, may be used to assess the cerebral hemorrhage and/or edema status of a subject and to evaluate a therapeutically effective amount of a binding-reduction molecule administered. Diagnostic tests for other these types of cerebral events are known to those of ordinary skill in the art. (for examples: see Harrison's Principles of Internal Medicine, 14/e, McGraw Hill companies, New York, 1998). A first determination of cerebral hemorrhage or edema may be obtained using one of the methods described above, and a subsequent determination of cerebral hemorrhage or edema can be done and a comparison of the presence or level of hemorrhage or edema may be used to assess the effectiveness of binding-reduction molecule administration as a prophylactic or a treatment of the cerebral hemorrhage or edema in a subject. Absence of a cerebral hemorrhage or edema may be an indication for prophylactic intervention by administering binding-reduction molecule to prevent hemorrhage or edema in a subject, for example in a subject know to be or believed to be likely to have a condition that may require thrombolytic treatment with tPA and/or uPA. The administration of tPA and/or uPA may also be an indication for co-treatment with a binding-reduction molecule of the invention. In addition, the administration of tPA and/or uPA may be an indication to follow such treatment with administration of a binding-reduction molecule of the invention.

The binding-reduction molecules may be administered alone, in combination with each other, and/or in combination with other drug therapies such as thrombolytic therapies and anti-hemorrhage or anti-edema therapies. These additional drug therapies and the parameters for their use will be known to those of skill in the art.

The above-described drug therapies are well known to those of ordinary skill in the art and are administered by modes known to those of skill in the art. The drug therapies are administered in amounts that are effective to achieve the physiological goals (to serve as a thrombolytic agent, or to reduce cerebral hemorrhage and/or edema), in combination with a binding-reduction molecule of the invention. Thus, it is contemplated that the drug therapies may be administered in amounts that are not capable of preventing or reducing the physiological consequences of the tPA and/or uPA side effects, e.g. cerebral hemorrhage or edema, when the drug therapies are administered alone, but which are capable of preventing or reducing the physiological consequences of the tPA and/or uPA side effects when administered in combination with the binding-reduction molecules of the invention.

When administered, the binding-reduction polypeptides or nucleic acids of the present invention are administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines and optionally other therapeutic agents. The term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art.

The therapeutics of the invention can be administered by any conventional route, including injection or by gradual infusion over time. The administration may, for example, be oral, intranasal, intravenous, intraperitoneal, intramuscular, intracavity, subcutaneous, or transdermal. An additional route of administration may be by pulmonary aerosol. Techniques for preparing aerosol delivery systems are well known to those of skill in the art. Generally, such systems should utilize components which will not significantly impair the biological properties of the medicament molecules, such as the protection against thrombolysis-associated reperfusion injury (see for example, “Aerosols,” in Remington's Pharmaceutical Sciences, 18th edition, 1990). Those of skill in the art can readily determine the various parameters and conditions for producing aerosols without resort to undue experimentation.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

The pharmaceutical compositions contain any of the foregoing therapeutic agents in a pharmaceutically acceptable carrier. Thus, in a related aspect, the invention provides a method for forming a medicament that involves placing a therapeutically effective amount of the therapeutic agent in the pharmaceutically acceptable carrier to form one or more doses.

The preparations of the invention are administered in effective amounts. An effective amount, as described above, is that amount of a pharmaceutical preparation that alone, or together with further doses, stimulates the desired response. In the case of treating or preventing side-effects of thrombolytic therapy, the desired response is inhibiting thrombolysis-associated reperfusion injury. This may involve only slowing the progression of the disorder temporarily, although more preferably, it involves halting the progression of the disorder permanently. These responses can be monitored by routine methods or can be monitored according to diagnostic methods of the invention discussed herein.

Compositions of the various forms of the binding-reduction nucleic acid molecules and polypeptides useful for practicing the invention are described herein. To accomplish the various treatments as described herein, a nucleic acid that encodes binding-reduction polypeptide or a functional portion or domain thereof is introduced into a mammalian cell (e.g., mammalian somatic cell, mammalian germ line cell (sperm and egg cells)). This can be accomplished by inserting the isolated nucleic acid that encodes either the full length binding-reduction polypeptide, the C-terminal domain, the N-terminal domain, or another domain, or a functional equivalent thereof, into a nucleic acid vector, e.g., a DNA vector such as a plasmid, virus or other suitable replicon (e.g., a viral vector), which can be present in a single copy or multiple copies. The nucleic acid can be transfected or transformed into cells using suitable methods known in the art such as electroporation, microinjection, infection, and lipofection and direct uptake. Such methods are described in more detail, for example, in Sambrook et al., “Molecular Cloning: A Laboratory Manual,” 2nd ED. (1989), Ausubel, F. M., et al., Current Protocols in Molecular Biology, (Current Protocol, 1994) and Sambrook et al., “Molecular Cloning: A Laboratory Manual,” 2nd, Ed. (1989).

A binding-reduction molecule of the invention can be used to prevent and/or treat a side effect of tPA and/or uPA thrombolytic treatment by delivering to cells the binding-reduction molecules, described herein, in vitro or in vivo. The cells to which binding-reduction molecules may be delivered in vitro include, but are not limited to, cultured cells. Examples of cultured cells to which the binding-reduction molecules of the invention may be delivered to prevent and/or treat a cerebral hemorrhage or edema, include but are not limited to, cells used to produce recombinant proteins and cells to be transplanted into a subject (e.g. bone marrow, blood cells, kidney cells, corneal cells, liver cells and stem cells).

Binding-reduction molecules can be delivered to a cell in vitro or in vivo by the use of viral vectors comprising one or more nucleic acid sequences encoding a binding-reduction polypeptide. Generally, the nucleic acid sequence has been incorporated into the genome of the viral vector. The viral vector containing nucleic acid sequences encoding the binding-reduction polypeptide can be contacted with a cell in vitro or in vivo and infection can occur. The cell can then be used experimentally to study, for example, the effect of binding-reduction molecules on tPA or uPA binding to their respective receptors in vitro or the cells can be implanted into a subject for therapeutic use. The cell can be migratory, such as hematopoietic cells, or non-migratory. The cell can be present in a biological sample obtained from the subject (e.g., blood, bone marrow) and used in the treatment of disease, or can be obtained from cell culture.

After contact with the binding-reduction polypeptide or with the viral vector comprising a nucleic acid sequence encoding the binding-reduction polypeptide, the subject sample can be returned to the subject or re-administered to a culture of subject cells according to methods known to those practiced in the art. In the case of delivery to a subject or experimental animal model (e.g., rat, mouse, monkey, chimpanzee), such a treatment procedure is sometimes referred to as ex vivo treatment or therapy. Frequently, the cell is taken from the subject or animal and returned to the subject or animal once contacted with the viral vector comprising the nucleic acids of the present invention. Ex vivo gene therapy has been described, for example, in Kasid, et al., Proc. Natl. Acad. Sci. USA 87:473 (1990); Rosenberg, et al., New Engl. J. Med. 323:570 (1990); Williams, et al., Nature 310476 (1984); Dick, et al., Cell 42:71 (1985); Keller, et al., Nature 318:149 (1985) and Anderson, et al., U.S. Pat. No. 5,399,346 (1994).

Where a cell is contacted in vitro, the cell incorporating the viral vector comprising a nucleic acid sequence of a binding-reduction polypeptide of the invention can be implanted into a subject or experimental animal model for delivery or used in in vitro experimentation to study cellular events mediated by the binding-reduction polypeptide.

Various viral vectors can be used to introduce the binding-reduction nucleic acid into mammalian cells. Viral vectors include retrovirus, adenovirus, parvovirus (e.g., adeno-associated viruses), coronavirus, negative-strand RNA viruses such as orthomyxovirus (e.g., influenza virus), rhabdovirus (e.g., rabies and vesicular stomatitis virus), paramyxovirus (e.g. measles and Sendai), positive-strand RNA viruses such as picomavirus and alphavirus, and double stranded DNA viruses including adenovirus, herpesvirus (e.g., Herpes Simplex virus types 1 and 2, Epstein-Barr virus, cytomegalovirus), and poxvirus (e.g. vaccinia, fowlpox and canarypox). Other viruses include Norwalk virus, togavirus, flavivirus, reoviruses, papovavirus, hepadnavirus, and hepatitis virus, for example. Examples of retroviruses include: avian leukosis-sarcoma, mammalian C-type, B-type viruses, D-type viruses, HTLV-BLV group, lentivirus, spumavirus (Coffin, J. M., Retroviridae: The viruses and their replication, In Fundamental Virology, Third Edition, B. N. Fields, et al., Eds., Lippincott-Raven Publishers, Philadelphia, 1996). Other examples include murine leukemia viruses, murine sarcoma viruses, mouse mammary tumor virus, bovine leukemia virus, feline leukemia virus, feline sarcoma virus, avian leukemia virus, human T-cell leukemia virus, baboon endogenous virus, Gibbon ape leukemia virus, Mason Pfizer monkey virus, simian immunodeficiency virus, simian sarcoma virus, Rous sarcoma virus, lentiviruses and baculoviruses.

A preferred method to introduce nucleic acid that encodes a binding-reduction into cells is through the use of engineered viral vectors. These vectors provide a means to introduce nucleic acids into cycling and quiescent cells, and have been modified to reduce cytotoxicity and to improve genetic stability. The preparation and use of engineered Herpes simplex virus type 1 (D. M. Krisky, et al., Gene Therapy 4(10):1120-1125.(1997)), adenoviral (A. Amalfitanl, et al., Journal of Virology 72(2):926-933. (1998)), attenuated lentiviral (R. Zufferey, et al., Nature Biotechnology 15(9)871-875 (1997)) and adenoviral/retroviral chimeric (M. Feng, et al., Nature Biotechnology 15(9):866-870 (1997)) vectors are known to the skilled artisan. In addition to delivery through the use of vectors, binding-reduction nucleic acids may be delivered to cells without vectors, e.g. as “naked” nucleic acid delivery using methods known to those of skill in the art.

Various forms of the binding-reduction polypeptides or nucleic acids of the invention, can be administered and delivered to a mammalian cell (e.g., by virus or liposomes, or by any other suitable methods known in the art or later developed). The method of delivery can be modified to target certain cells, and in particular, cell surface receptor molecules or antigens present on tumor cells. Methods of targeting cells to deliver nucleic acid constructs are known in the art. The binding-reduction polypeptides can also be delivered into cells by expressing a recombinant protein fused with peptide carrier molecules, examples of which, though not intended to be limiting, are tat or antennapedia. These delivery methods are known to those of skill in the art and are described in U.S. Pat. No. 6,080,724, and U.S. Pat. No. 5,783,662, the entire contents of which are hereby incorporated by reference.

In addition to the methods described herein for delivering exogenous binding-reduction molecules, expression of endogenous binding-reduction polypeptides can be induced (e.g. upregulated) in cells harboring the virus by the administration of chemicals or other molecules that specifically increase the level of binding-reduction mRNA and/or protein expression. Such induction and/or upregulation of endogenous binding-reduction molecules may occur through methods that include, but not limited to: (a) activation of the binding-reduction molecule promoter, (b) stabilization of binding-reduction mRNA, (c) increased translation of a binding-reduction polypeptide and (d) stabilization of a binding-reduction polypeptide.

A binding-reduction polypeptide may be administered using other methods known in the art. For example, the mode of administration is preferable at the location of the target cells. Other modes of administration (parenteral, mucosal, systemic, implant, intraperitoneal, etc.) are generally known in the art. The agents are preferably administered in a pharmaceutically acceptable carrier, such as saline, sterile water, Ringer's solution, and isotonic sodium chloride solution.

In another aspect of the invention includes methods and compositions that are useful to modulate binding of tPA to LRP or modulate binding of uPA to uPAR in a way that increases the level of binding to a level above a control level. These compositions may include agents that are binding-increasing molecules. The description provided above here relating to the binding-reduction molecules is also applicable to the binding-enhancing molecules, is so far as the types of molecules that may be used, but the effect of the binding-enhancing molecules is to increase the level of binding of tPA and uPA to LRP and uPAR respectively, thereby increasing the level of MMP or proteases produced. Such methods and compositions may be used to produce models (cell, tissue, and animal models) of cerebral conditions using methods known to those in the art. These enhancing models may be used to examine the effects of drugs and treatments on cerebral conditions such as cerebral hemorrhage and/or edema.

In addition to the methods and compositions of the invention that are directed toward preventing and treating reperfusion injury, the invention also includes diagnostic methods and compositions. Some subjects may have LRP/uPAR and/or MMP/other protease genes that are more responsive to tPA/uPA-induced protease dysregulation. These subjects may be less eligible for thrombolytic therapy vis-à-vis risk/benefit considerations. The diagnostic methods include the determination of the effect on MMP levels in subjects to whom tpA and/or uPA is administered. Such methods include determining the effect or level of the effect of tPA/LRP receptor binding and/or uPA/uPAR binding on the dysregulation of MMP or related proteases in a cell or tissue from a subject. The cells and/or tissues may be cultured or freshly obtained through biopsy.

The diagnostic methods of the invention may be useful to tailor or determine eligibility for use of a thrombolytic regimen for individual subjects. For example, a determination that upon administration of uPA and/or tPA a subject has a level of MMP upregulation that is significantly higher than a control level of MMP upregulation, may contraindicate the use of thrombolytic therapy in the subject, or may indicate a need to administer a higher dose of one or more of the compositions of the invention that reduce side effects of tPA or uPA administration. Thus, the diagnostic methods are useful to allow health-care providers to tailor thrombolytic therapy regimens for individual subjects and/or patients.

The invention will be more fully understood by reference to the following examples. These examples, however, are merely intended to illustrate the embodiments of the invention and are not to be construed to limit the scope of the invention.

### EXAMPLES

### Example 1

Inhibition of MMP Reduces tPA-Induced Hemorrhage in Rat Embolic Focal Ischemia

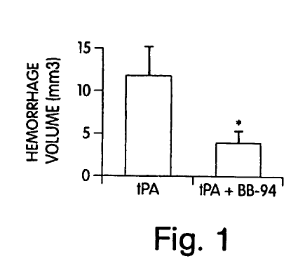

The involvement of MMPs in hemorrhagic transformation was investigated because these proteases can degrade matrix substrates that weaken blood vessel integrity. The quantitative rat model of tPA-induced hemorrhage that we developed was used to examine the role of MMPs in hemorrhagic transformation. Rats were subjected to embolic (clot-based) focal ischemia, then treated with either tPA alone (10 mg/kg iv, 6 hrs post-ischemia) or with tPA plus two doses of 50 mg/kg ip of the broad spectrum MMP inhibitor batimastat (BB-94, which was donated by British Biotech, Oxford, G.B.). Hemorrhage volumes were quantified at 24 hrs with hemoglobin spectrophotometry in perfused brain. tPA-induced hemorrhage volumes were significantly decreased by co-treatment with BB-94 (FIG. 1). These data indicated that MMPs mediate tPA-induced hemorrhagic transformation after ischemic stroke (Sumii, T., and E. H. Lo, Stroke 33:831-836, 2002).

Addition of tPA Amplifies MMP-9 Levels after Focal Ischemia in Vivo

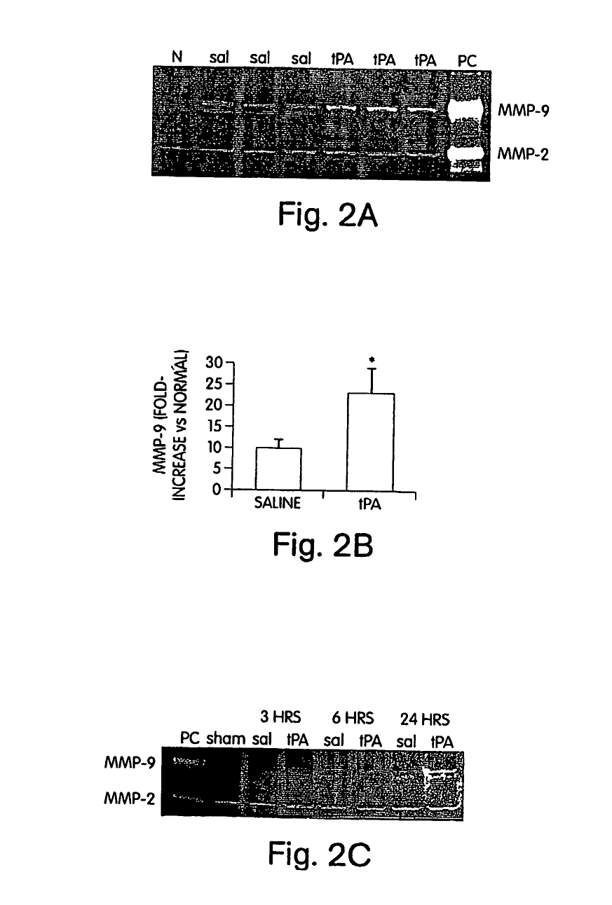

The use of BB-94 provided pharmacologic evidence that MMPs were involved in tPA-induced hemorrhage. The role of tPA in further increasing MMP levels was investigated (Sumii, T, E. H. Lo, Stroke 33:831-836, 2002; Aoki, T., et al., Stroke 33:2711-2717, 2002). At 12 hrs after embolic focal ischemia in rats, gelatin zymograms show an upregulation in MMP-9, as expected (FIG. 2A). However, rats that were treated with tPA (10 mg/kg, 6 hrs post-ischemia) showed even higher MMP-9 levels (FIG. 2A). This is an important point since MMP-9 is already produced in ischemic brain. The relevance of tPA-induced MMP-9 is dependent on how much more is released. The results demonstrated that tPA more than doubled the production of MMP-9 in our model. MMP-2 levels were also slightly increased but the difference did not reach statistical significance within the limits of the number of data points available (FIG. 2B).

Similar findings were obtained in mechanical (filament) models of focal ischemia. Rats were subjected to transient occlusions for 2 hrs, then reperfused for 3, 6, or 24 hrs. Saline controls were compared with rats treated with 10 mg/kg of tPA at the onset of reperfusion. Treatment with tPA clearly amplified the upregulation of MMP-9 as measured on the gelatin zymograms (FIG. 2C).

Knockout of tPA Gene Reduces MMP-9 Upregulation after Focal Cerebral Ischemia

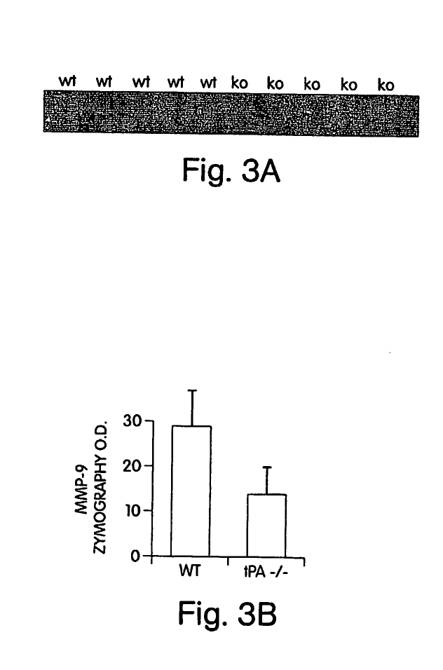

To support the pharmacologic data shown above, permanent focal ischemia was induced in tPA knockout mice and wild-type mice. At 24 hrs, MMP-9 levels in ischemic brain were elevated in wild-type mice. However, MP-9 levels were significantly lower in the tPA knockout mice (FIG. 3). These data indicated that tPA upregulates deleterious MMP-9 levels after cerebral ischemia.

Knockout of MMP-9 Gene Reduces Infarction and Edema after Focal Cerebral Ischemia

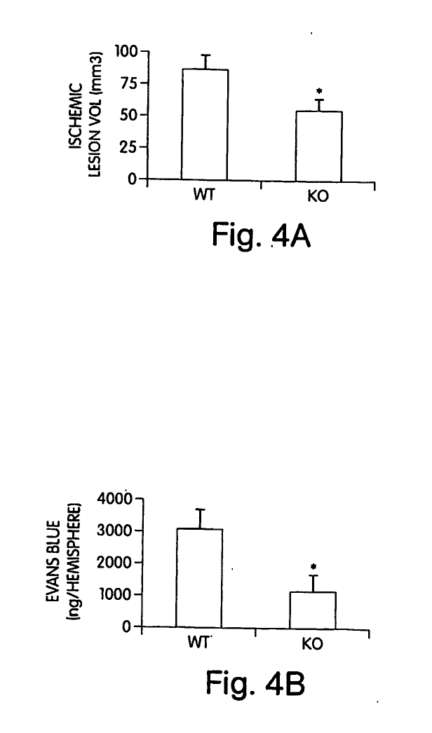

We investigated whether MMP-9 knockouts would also have less blood-brain barrier disruption and overall brain damage (Asahi, M., et al., J Cereb Blood Flow Metab 20:1681-1690, 2000; Ashi, M., et al., J Neurosci 21:7724-7732, 2001). MMP-9 knockouts and wild-type littermates were subjected to 2 hr transient focal ischemia. At 24 hrs, ischemic lesion volumes were significantly smaller in knockouts compared to wild-type mice (FIG. 4A). In addition, Evans blue leakage at 20 hrs after reperfusion were also significantly reduced in MMP-9 knockout mice (FIG. 4B).

LRP is Upregulated in Neurons, Astrocytes, and Brain Endothelial Cells after Hypoxia in Vitro

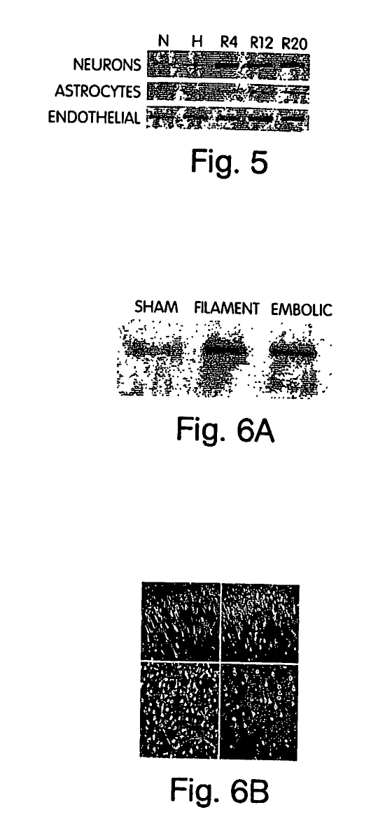

To examine whether the binding of tPA onto the LRP receptor upregulates signaling pathways that lead to secretion of neurovascular matrix-damaging proteases from the MMP family the following examinations were performed. LRP was detected on cells of the neurovascular unit, i.e. cerebral endothelial cells, astrocytes, and neurons. Western blots showed a clear band for LRP, which were increased after hypoxia in vitro (FIG. 5).

LRP is Upregulated in Rat Brain after Focal Ischemia in Vivo

Brain tissue was examined for expression of LRP in vivo, and LRP was detected in vivo. Rats were subjected to mechanical (filament) and embolic focal ischemia. Western blots and immunohistochemistry showed that LRP was increased after ischemia (FIG. 6A and FIG. 6B).

Addition of tPA to in Vitro Cultures of Neurons, Astrocytes and Cerebral Endothelial Cells Induce Secretion of MMP-2 and MMP-9

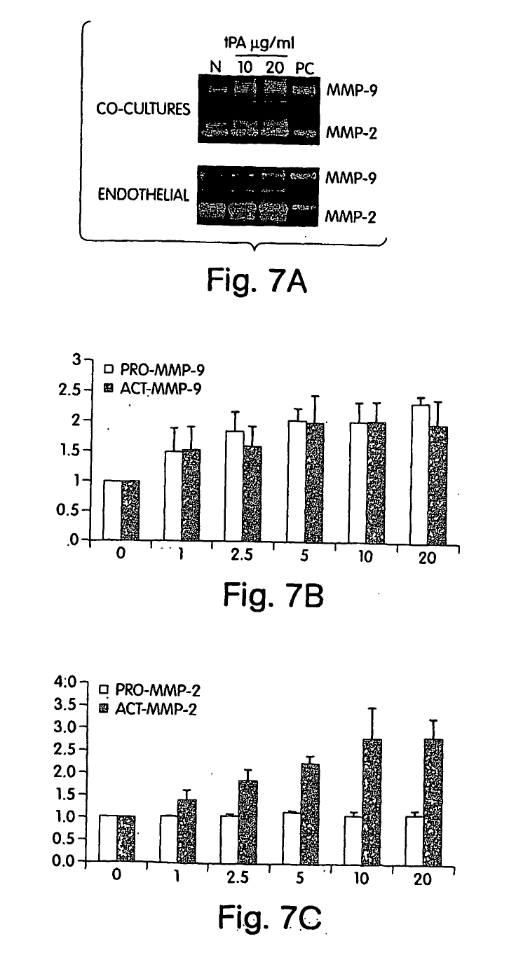

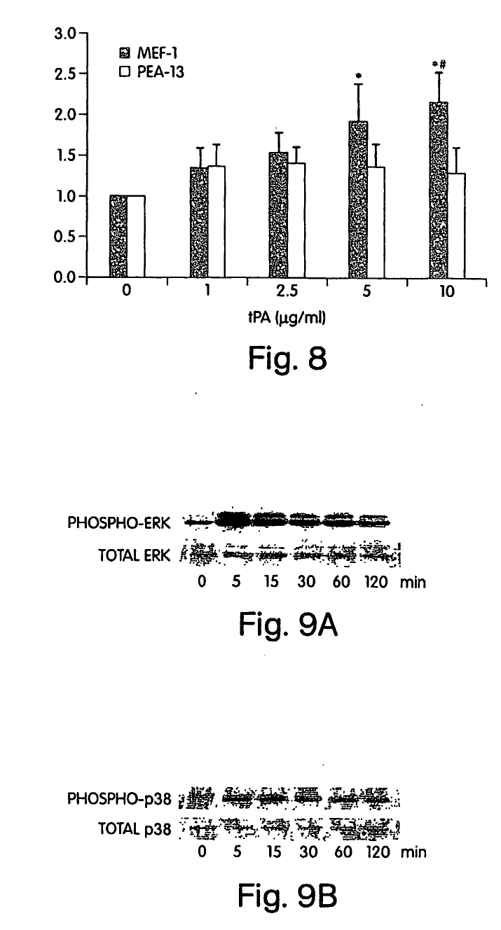

Different doses of tPA were added to primary co-cultures of neurons/astrocytes from rat brain, or microvessel endothelial cells from human brain. Cytotoxicity assays of LDH release confirmed that these doses were not toxic, consistent with our previous published experience using tPA in vitro (Wang X, et al., Neurosci Lett 274:79-82, 1999). In addition, these doses are within range of plasma concentrations in patients treated with intravenous tPA (NINDS rt-PA Stroke Study Group, New Engl J Med 333:1581-1587, 1995). Conditioned media was then analyzed using zymography to detect MMP-2 and MMP-9. tPA clearly induced secretion of MMP-9 in both the neuron/astrocyte co-cultures and the endothelial cells (FIG. 7A). There was also a clear dose- and time-dependence in the tPA-induced MMP-2 and MMP-9 release (FIG. 7B and FIG. 7C).

Genetic Knockout of LRP Eliminates the tPA-Induced MMP-9 Response

To determine whether tPA-induced MMP-9 production is mediated by binding to LRP, murine embryonic fibroblasts (MEF) cells either from wild-type mice (MEF-1) or knockout mice lacking LRP expression (PEA-13) were tested. LRP knockout cells showed no tPA-induced MMP-9 response, confirming that tPA-induced MMP upregulation was mediated via the LRP receptor.

MAP Kinase Pathways are Upregulated in Neurons after tPA Exposure in Vitro

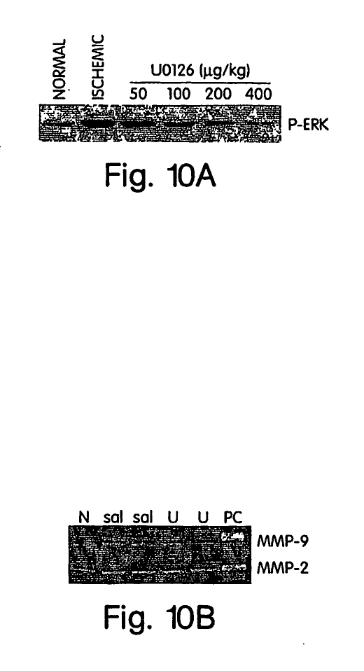

The presence of MMP upregulation following activation of various signaling pathways after tPA binds to LRP, was investigated. In this investigation, MAP kinase was examined. In rat astrocyte cultures, there was activation of ERK (FIG. 9A) and p38 MAP kinase (FIG. 9B) pathways after addition of tPA.

Inhibition of ERK MAP Kinase Reduces MMP-9 in Vivo

We investigated whether inhibition of MAP kinase signaling pathways reduced the upregulation of MMPs. In this investigation, rats were pre-treated with either saline or U0126 intravenously, then subjected to 2 hr focal ischemia. U0126 inhibits MEK, which is the upstream kinase that activates ERK. Western blots showed that U0126 significantly inhibited ERK MAP kinase activation (FIG. 10A). The resulting data also indicated that U0126 can attenuate ischemic upregulation of MMP-9 (FIG. 10B). Similar reductions of MMP-9 by ERK inhibition has been shown in a mouse brain trauma model (Mori T., et al., J. Neurotrauma 19(11):1411-1419, 2002).

### EQUIVALENTS

Those skilled in the art will recognize, or be able to ascertain using no more than routine experimentation, many equivalents to the specific embodiments of the invention described herein. Such equivalents are intended to be encompassed by the following claims.

All references, including patent documents, disclosed herein are incorporated by reference in their entirety.

We claim:

1. A method for reducing a side effect associated with thrombolytic therapy, comprising
inhibiting binding of tissue plasminogen activator (tPA) administered to a subject to a low-density lipoprotein-receptor-related protein (LRP) receptor.

2. The method of claim 1, wherein inhibiting binding of tPA to LRP comprises administering to a subject in need of such treatment an amount of an agent that reduces tissue plasminogen activator (tPA) binding to a low-density lipoprotein-receptor-related protein (LRP) receptor effective to reduce the side effect, wherein the agent is administered before, simultaneously with, or after tPA treatment.

3. The method of claim 1, wherein the side effect associated with thrombolytic therapy is cerebral hemorrhage and/or edema.

4. The method of claim 1, wherein the subject is human.

5. The method of claim 1, wherein the thrombolytic therapy is the administration of tPA.

6. The method of claim 2, wherein the agent that reduces tPA binding to a LRP receptor is administered before tPA treatment.

7. The method of claim 2, wherein the agent that reduces tPA binding to a LRP receptor is administered simultaneously with tPA treatment.

9. The method of claim 2, wherein the administration is intravenous administration.

10. The method of claim 2, wherein the agent is an antibody or antigen-binding fragment thereof.

11. The method of claim 1, wherein the subject is suspected or known to be at risk for a condition selected from the group consisting of ischemia, hemorrhage, edema, and brain injury.

12. The method of claim 1, wherein the subject is suspected or known to have a condition selected from the group consisting of: ischemia, hemorrhage, edema, and brain injury.

13. The method of claim 1, wherein the subject is suspected or known to have had a condition selected from the group consisting of: ischemia, hemorrhage, edema, and brain injury.

14-28. (canceled)

29. A method of identifying a candidate agent that modulates tissue plasminogen activator (tPA) binding to a low-density lipoprotein-receptor-related protein (LRP) receptor comprising:
contacting an LRP receptor with tPA in the presence of a candidate agent,
determining the level of binding of the LRP receptor with the tPA, and
comparing the level of binding of LRP with tPA with a control level of binding of LRP and tPA not contacted with the candidate agent as a measure of the ability of the candidate agent to modulate tPA binding to LRP receptor.

30. The method of claim 29, wherein modulate is to reduce.

31. The method of claim 29, wherein modulate is to increase.

30. The method of claim 29, wherein modulate is to reduce.

31. The method of claim 29, wherein modulate is to increase.

32. The method of claim 29, wherein the tPA is labeled with a detectable label.

33. The method of claim 29, wherein the LRP receptor is labeled with a detectable label.

34-41. (canceled)

42. A method of identifying a thrombolytic tissue plasminogen activator (tPA) variant with reduced binding to a low-density lipoprotein-receptor-related protein (LRP) receptor, comprising:
modifying a tPA molecule to prepare modified tPA molecules,
testing the thrombolytic activity of the modified tPA molecules,
selecting modified tPA molecules that retain thrombolytic activity (modified thrombolytic tPA molecules),
contacting an LRP receptor with the modified thrombolytic tPA molecules,
determining the level of binding of the LRP receptor with modified thrombolytic tPA molecules, and
comparing the level of binding of LRP receptor by modified thrombolytic tPA molecules with a control level of binding of LRP receptor by unmodified tPA as an indication of reduced binding of the modified thrombolytic tPA molecules to LRP receptor.

43. The method of claim 42, wherein the modification of the tPA molecule comprises one or more modifications selected from the group consisting of amino acid substitutions, amino acid deletions, and post-translational modifications.

44-45. (canceled)
